(19) United States
(12) Patent Application Publication
(10) Pub. No.: US 2023/0026776 A1
(43) Pub. Date: Jan. 26, 2023

(54) IMPLANTABLE SELF-CLOSING CUFF

(71) Applicant: INCUBE LABS, LLC, San Jose, CA (US)

(72) Inventors: Paul SPEHR, San Antonio, TX (US); Charles Gregory NELSON, New Braunfels, TX (US); Varghese K. GEORGE, San Jose, CA (US); Isabell PINA, San Antonio, TX (US)

(73) Assignee: INCUBE LABS, LLC, San Jose, CA (US)

(21) Appl. No.: 17/787,148

(22) PCT Filed: Oct. 15, 2020
(86) PCT No.: PCT/US2020/055770

§ 371 Date: Jun. 17, 2022

Related U.S. Application Data
(60) Provisional application No. 62/951,981, filed on Dec. 20, 2019.

(51) Int. Cl. A41B 7/02

(52) U.S. Cl. CPC A41B 7/02

(57) ABSTRACT

A cuff is described for a target anatomic feature within a body, along with a system for utilizing the cuff. The cuff includes a band defining a circumferential opening extending along a length of the band; and a pair of engagement surfaces defined by or affixed to the cuff, the engagement surfaces structured for application of a spreading force to be distributed continuously along a portion of the circumferential opening, thereby increasing the circumferential opening and expanding the cuff for a closed configuration to an open configuration sized for placement of the cuff. The cuff is structured to remain in the closed configuration in the absence of the spreading force and automatically return to the closed configuration after removal of the spreading force.

### CROSS-REFERENCE TO RELATED APPLICATIONS

This application claims priority to, and the benefit of, U.S. Provisional Patent Application No. 62/951,981 filed on Dec. 20, 2019 and titled SELF-CLOSING CUFF ELECTRODE AND METHOD OF INSTALLATION, which is incorporated herein by reference in its entirety for all purposes.

### STATEMENT REGARDING FEDERALLY SPONSORED RESEARCH OR DEVELOPMENT

This invention was made with Government support under W81XWH-15-C-0066, awarded by U.S. Department of Defense, U.S. Army Medical Research Acquisition Activity. The Government has certain rights in the invention.

### BACKGROUND

There has been an increase in the use of various implantable medical treatment devices. For example, a medical treatment device or a portion thereof may be positioned at a target region within a body to apply a treatment. A relatively precise positioning of the medical treatment device with respect to the target region may be important for a treatment to be effective. However, precise initial positioning may be challenging, and maintaining the precise positioning over time may pose an additional challenge. For example, it may be desirable in certain medical applications for the medical treatment device to be maintained in the body for days, weeks or even indefinitely, depending on the treatment provided. The body may react over time to the presence of the medical treatment device, such as by movement or settlement of tissue surrounding the device, fluid build-up, and/or formation of scar tissue. Such reactions can cause movement (e.g., migration) of the device, which can result in a reduction in effectiveness of the treatment, or delivery of the treatment to the wrong site.

The target region may be, or may be adjacent to, an anatomic feature having a curved outer surface such as a nerve, an artery, a vein, a luminal organ, a bone, a tendon, a ligament, musculature, or a fascia. For such regions, a component of the medical treatment device may be used to circumscribe the anatomic feature to initially position the device and then subsequently hold the device in position. The component may be a cuff. A conventional means for securing and maintaining a cuff or other component is to place a suture around the component. This technique relies on the skill of the person doing the suturing to apply an appropriate amount of tension on the suture. If the suture is too tight, the anatomic feature may be damaged. If the suture is too loose, the medical treatment device may not make adequate contact with the anatomic feature, and thus may fail to provide effective treatment, and/or may deliver treatment to non-target or surrounding tissue.

### BRIEF SUMMARY

Embodiments provide medical treatment devices and methods for delivering treatment to a target region within a body. Additionally, a self-closing cuff is described that is particularly suited for coupling to anatomic features with curved outer surfaces, and a placement tool is described for positioning the self-closing cuff with respect to the anatomic feature.

The self-closing cuff may be a cuff electrode structured to provide electrical stimulation to a target region such as a nerve. The cuff electrode may be structured to be coupled to a stimulator, which may be implanted in a body, or may be external to the body.

The self-closing cuff may be attached to a housing of an implantable medical treatment device. The self-closing cuff may be attached to a catheter or cannula.

A medical treatment device can be introduced into an animalia body, and a self-closing cuff of the medical treatment device can be used to position a treatment portion of the medical treatment device at a target treatment location within the body. A placement tool may be used to open the self-closing cuff to position the cuff on the anatomic feature. The placement tool is then removed, leaving the cuff.

Further aspects of the technology described herein will be brought out in the following portions of the specification, wherein the detailed description is for the purpose of fully disclosing embodiments of the technology without placing limitations thereon.

### BRIEF DESCRIPTION OF THE DRAWINGS

The technology described herein will be more fully understood by reference to the following drawings which are for illustrative purposes only:

FIG. 1 illustrates an example of an embodiment of a self-closing cuff including electrodes.

FIG. 2 illustrates an example of an embodiment of a self-closing cuff including electrodes.

FIG. 3 illustrates an example of an embodiment of the cuff of FIG. 1.

FIG. 4 illustrates an example of an embodiment of the cuff of FIG. 2.

FIG. 5 illustrates an example of an embodiment of a retainer for incorporation with a self-closing cuff.

FIG. 6 illustrates an example of an embodiment of a self-closing cuff including suture through-holes.

FIG. 7A illustrates an example of an embodiment of a self-closing cuff including a suture tube.

FIG. 7B illustrates an example of an embodiment of the self-closing cuff of FIG. 7A.

FIG. 8A illustrates an example of an embodiment of a placement tool in a closed configuration.

FIG. 8B illustrates an example of an embodiment of the placement tool of FIG. 8A in an open configuration.

FIG. 9 illustrates a perspective view of an example of an embodiment of a self-closing cuff positioned to be received on the placement tool of FIGS. 8A, 8B.

FIG. 10 illustrates a perspective view of using the placement tool of FIGS. 8A, 8B to position a self-closing cuff in a body.

FIG. 11 illustrates an example of an embodiment of a self-closing cuff received on an embodiment of a placement tool.

FIG. 12A illustrates an example of an embodiment of the placement tool of FIG. 11.

FIG. 12B illustrates an example of an embodiment of the self-closing cuff of FIG. 11.

FIG. 13 illustrates an example of an embodiment of a self-closing cuff received on an embodiment of a placement tool.

FIG. 14A and FIG. 14B illustrate an example of an embodiment of the placement tool of FIG. 13, in a closed configuration and in an open configuration, respectively.

### DETAILED DESCRIPTION

Embodiments provide medical treatment devices and methods for delivering treatment to a target region within a body. Additionally, a self-closing cuff is described that is particularly suited for coupling to anatomic features with curved outer surfaces, and a placement tool is described for positioning the self-closing cuff with respect to the anatomic feature.

In an embodiment, a self-closing cuff is a cuff electrode structured to provide electrical stimulation to a target region such as a nerve. The cuff electrode may be structured to be coupled to a stimulator, which may be implanted in a body, or may be external to the body.

In an embodiment, a self-closing cuff is attached to a housing of an implantable medical treatment device. In an embodiment, a self-closing cuff is attached to a catheter or cannula.

In an embodiment of a medical treatment application or process, a medical treatment device can be introduced into an animalia body, and a treatment portion of the medical treatment device positioned at a target treatment location within the body. The introduction of the medical treatment device can be performed by a doctor or medical professional, often through the use of an imaging modality (e.g., fluoroscopy, ultrasound, endoscopic image capture, or other imaging technique) or using one or more sensing devices that allow attending personnel to position the medical treatment device relatively precisely. It may then be desirable for the medical treatment device to retain its position for days, weeks, months or years with little movement, to improve eventual success of the treatment being applied.

A placement tool may be used to open a self-closing cuff to deliver the cuff on the anatomic feature. The placement tool is then removed.

As an example of an embodiment of a medical treatment application or process, a self-closing cuff is a cuff electrode that may encircle or partially encircle an external curved surface of an anatomic feature to provide electrical stimulation to, or adjacent to, the anatomic feature. Such electrical stimulation may be applied, for example, to a nerve (e.g., for pain management, for blocking of signal conduction along the nerve to avoid a responsive action by the body, or for inducing signal conduction along the nerve to induce a responsive action by the body), or to a muscle (e.g., to allow or to inhibit muscle contraction). In an embodiment, a stimulation controller in electrical communication with the cuff electrode may provide energy for, and control of, electrical stimulation provided by the cuff electrode.

In an embodiment, a self-closing cuff electrode is provided for use in a treatment system for control of urination (e.g., initiating and/or terminating urination) by stimulation of one or more nerves, such as including stimulation of a pudendal nerve. Other treatment locations suitable for a self-closing cuff electrode for stimulation of nerves include, for example, nerves leading to or within the spinal cord, or nerves in association with heart, stomach, bladder, or other organ.

While embodiments of the present disclosure are particularly suited for self-closing cuff electrodes for providing stimulation to nerves, it is appreciated that a self-closing cuff may be used on a variety of different anatomic features, and used for a number of different treatment and/or detection modalities. For example, a self-closing cuff may position a catheter or cannula to deliver a medication at a particular treatment site, to position a sensor at a particular detection site, to position a thermal device at a particular treatment site, to position a vibratory or ultrasonic device at a particular treatment site, to reposition one anatomic feature with respect to another anatomic feature, or to couple two anatomic features together. Accordingly, embodiments of a self-closing cuff may be used to interface with an artery, a vein, a luminal organ, a bone, a tendon, a ligament, the urethra, musculature, a fascia, or other anatomic feature.

Having provided an overview, some examples of embodiments illustrated by the figures will be described in detail. First, however, some conventions are provided for the convenience of the reader.

Various abbreviations may be used herein for standard units, such as deciliter (dl), milliliter (ml), microliter (μl), international unit (IU), centimeter (cm), millimeter (mm), nanometer (nm), inch (in), kilogram (kg), gram (gm), milligram (mg), microgram (μg), millimole (mM), degrees Celsius (° C.), degrees Fahrenheit (° F.), millitorr (mTorr), hour (hr), or minute (min). Also used herein is the standard unit mmHg, which refers to a unit of pressure equal to the pressure exerted by a column of mercury 1 millimeter high at 0° C. and under the acceleration of gravity.

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural referents unless the context clearly dictates otherwise. Reference to an object in the singular is not intended to mean “one and only one” unless explicitly so stated, but rather “one or more.”

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be omitted or can be used in another embodiment (e.g., in another embodiment illustrated and described herein, or in another embodiment within the scope of the present disclosure and not illustrated and/or not described herein).

The term “component” refers herein to one item of a set of one or more items that together make up a device, formulation or system under discussion. A component may be in a solid, powder, gel, plasma, fluid, gas, or other form. For example, a device may include multiple solid components which are assembled together to structure the device and may further include a liquid component that is disposed in the device. For another example, a formulation may include two or more powdered and/or fluid components which are mixed together to make the formulation.

The term “design” or a grammatical variation thereof (e.g., “designing” or “designed”) refers herein to characteristics intentionally incorporated based on, for example, estimates of tolerances (e.g., component tolerances and/or manufacturing tolerances) and estimates of environmental conditions expected to be encountered (e.g., temperature, humidity, external or internal ambient pressure, external or internal mechanical pressure, stress from external or internal mechanical pressure, age of product, or shelf life, or, if introduced into a body, physiology, body chemistry, biological composition of fluids or tissue, chemical composition of fluids or tissue, pH, species, diet, health, gender, age, ancestry, disease, or tissue damage); it is to be understood that actual tolerances and environmental conditions before and/or after delivery can affect characteristics so that different components, devices, formulations, or systems with a same design can have different actual values with respect to those characteristics. Design encompasses also variations or modifications before or after manufacture.

The term “manufacture” or a grammatical variation thereof (e.g., “manufacturing” or “manufactured”) as related to a component, device, formulation, or system refers herein to making or assembling the component, device, formulation, or system. Manufacture may be wholly or in part by hand and/or wholly or in part in an automated fashion.

The term “structured” or a grammatical variation thereof (e.g., “structure” or “structuring”) refers herein to a component, device, formulation, or system that is manufactured according to a concept or design or variations thereof or modifications thereto (whether such variations or modifications occur before, during, or after manufacture) whether or not such concept or design is captured in a writing.

The term “body” refers herein to an animalia body. The term “subject” refers herein to a body into which an embodiment of the present disclosure is, or is intended to be, delivered. For example, with respect to humans, a subject may be a patient under treatment of a health care professional.

The terms “substantially” and “about” are used herein to describe and account for small variations. For example, when used in conjunction with a numerical value, the terms can refer to a variation in the value of less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

As used herein, a range of numbers includes any number within the range, or any sub-range if the minimum and maximum numbers in the sub-range fall within the range. Thus, for example, “<9” can refer to any number less than nine, or any sub-range of numbers where the minimum of the sub-range is greater than or equal to zero and the maximum of the sub-range is less than nine. Ratios may also be presented herein in a range format. For example, a ratio in the range of about 1 to about 200 should be understood to include the explicitly recited limits of about 1 and about 200, and also to include individual ratios such as about 2, about 35, and about 74, and sub-ranges such as about 10 to about 50, about 20 to about 100, and so forth.

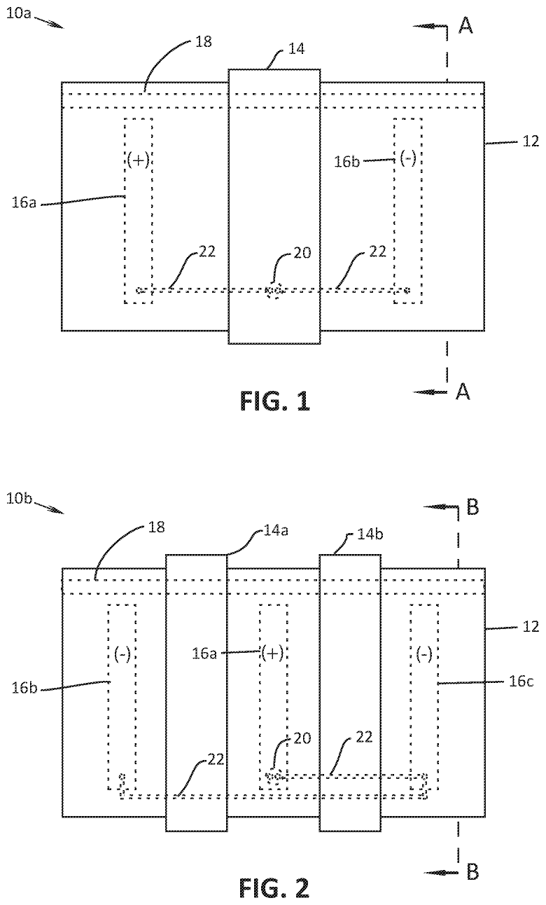

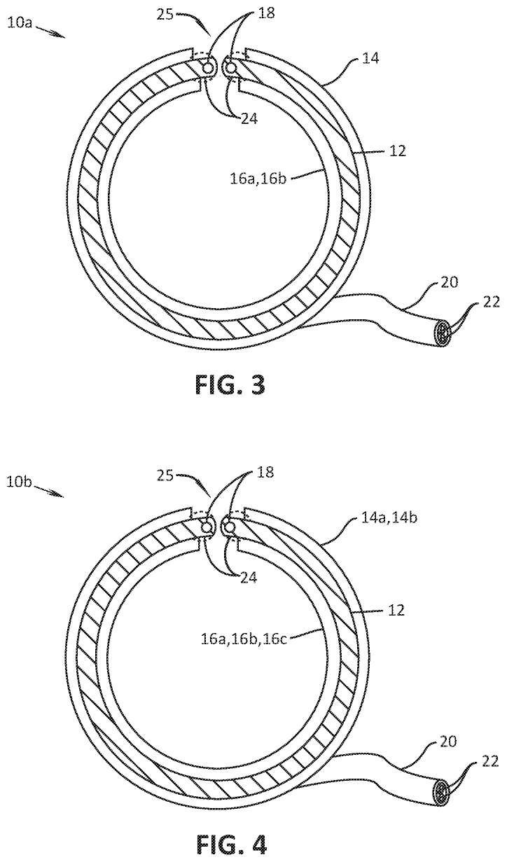

Attention is now drawn to the figures. FIG. 1 through FIG. 4 illustrate embodiments of a self-closing cuff 10. Specifically, FIG. 1 illustrates an embodiment of a self-closing cuff 10a (an embodiment of the self-closing cuff 10) and FIG. 3 illustrates an embodiment of the self-closing cuff 10a of FIG. 1; and FIG. 2 illustrates an embodiment of a self-closing cuff 10b (an embodiment of the self-closing cuff 10) and FIG. 4 illustrates an embodiment of the self-closing cuff 10b of FIG. 2.

FIG. 1 illustrates a side view of the self-closing cuff 10a. The cuff 10a includes a band 12 having a central channel defined by an inner circumferential surface of the band 12.

The cuff 10a further includes a pair of longitudinal apertures 18. In the embodiment of FIG. 1, the longitudinal apertures 18 are shown extending along an entirety of the length of the cuff 10a; the longitudinal apertures 18 may alternatively extend along a portion of the length of the cuff 10a that is less than an entirety of the length of the cuff 10a. Further, the cuff 10a may include two or more pairs of the longitudinal apertures 18. In an embodiment, the cuff 10a includes two or more pairs of the longitudinal apertures 18 and central axes of at least two of the longitudinal apertures 18 falls approximately along a same line. In an embodiment, the cuff 10a includes two or more pairs of the longitudinal apertures 18 and central axes of at least two of the longitudinal apertures 18 are substantially parallel.

FIG. 3 is a section view of an embodiment of the cuff 10a of FIG. 1 (e.g., along section line A-A in FIG. 1). In the embodiment of FIG. 3, the band 12 includes opposing ends 24 which extend longitudinally along the length of the band 12 to define a circumferential opening 25. The circumferential opening 25 allows the cuff 10a to be opened and closed for coupling and/or decoupling of the cuff 10a to a target anatomic feature. The circumferential opening 25 may be structured such that the opposing ends 24 touch in a closed configuration, or such that the opposing ends 24 do not touch in the closed configuration and a gap remains between the opposing ends 24 in the closed configuration.

In the embodiments of FIG. 1 and FIG. 3, a retainer 14 is disposed on an outer circumference of the band 12. The retainer 14 has a material selection, size and shape to provide a designed closing force to the cuff 10a from an open configuration of the cuff 10a, for optimal retention of the cuff 10a at or around an anatomic feature where the cuff 10a is to be positioned. The closing force is designed, for example, considering a size and sensitivity of the target anatomic feature as well as a pressure expected to be needed to be exerted against the target anatomic feature for efficacy of a treatment and/or sufficiency of retention of the cuff 10a in a desired position with respect to the target anatomic feature. In an embodiment, the retainer 14 is designed to apply a specified force to a nerve (the anatomic feature in this embodiment) so as not to damage or alter function of the nerve as a result of the specified force, while still retaining an electrode of the cuff 10a in a desired position with respect to the nerve. The closing force of the retainer 14 is further designed to allow the cuff 10a to be opened with application of a spreading force by a person installing the cuff 10a at the target anatomic feature.

In an embodiment, the retainer 14 includes an approximately c-shaped flat ribbon spring.

While the embodiments of FIG. 1 and FIG. 3 illustrate a single retainer 14 approximately centrally located along a long axis of the band 12, it is appreciated that varying numbers and locations of the retainers 14 may be implemented as appropriate according to a size and/or shape of the cuff 10a and the size and/or shape of the target anatomic feature.

In an embodiment, the cuff 10a is a bi-polar cuff electrode including two electrodes 16 (shown as electrodes 16a, 16b) housed within or on the inner circumferential surface of the band 12 at axially spaced-apart locations along the band 12. By way of example in the embodiment of FIG. 1, the electrode 16a is shown as having a positive polarity and the electrode 16b is shown as having a negative polarity.

If the electrodes 16a, 16b are present in an embodiment, electrical conduits 22 that preferably pass through one or more openings in the band 12 are electrically coupled to the electrodes 16a, 16b (e.g., by thermal or vibration welding, or soldering) and are further electrically coupled to electronics (not shown) that provide electrical energy through the electrical conduits 22 to the electrodes 16a, 16b. Such electronics may be on the cuff 10a or apart from the cuff 10a (either internal or external to the body). Each of the electrical conduits 22 may be, for example, implemented as a wire or wires (e.g., single strand wire, or multi-strand wire, and the electrical conduits 22 may be, for example, twisted, helically arranged, or braided), or implemented as an electrical trace or traces (e.g., flat conductors or printed conductive traces), or a combination thereof. Each of the electrical conduits 22 may include more than one type of conductor, such as a printed trace within the cuff 10a and a wire or wires extending from outside of the band 12 to the electronics. The electrical conduits 22 or portions thereof may be contained in a flexible sleeve 20.

FIG. 2 illustrates a side view of an embodiment of the self-closing cuff 10b. The cuff 10b of FIG. 2 is similar to the cuff 10a of FIG. 1, with a distinction being that the cuff 10b of FIG. 2 includes two retainers 14 (shown as retainers 14a, 14b). The retainers 14a, 14b separately and/or collectively have a material selection, size and shape to provide a designed closing force to the cuff 10a from an open configuration of the cuff 10a, for optimal retention of the cuff 10a at or around an anatomic feature where the cuff 10a is to be positioned. The closing force is designed, for example, considering a size and sensitivity of the target anatomic feature as well as a pressure expected to be needed to be exerted against the target anatomic feature for efficacy of a treatment and/or sufficiency of retention of the cuff 10a in a desired position with respect to the target anatomic feature. In an embodiment, the retainers 14a, 14b are separately and/or collectively designed to apply a specified force to a nerve (the anatomic feature in this embodiment) so as not to damage or alter function of the nerve as a result of the specified force, while still retaining an electrode of the cuff 10a in a desired position with respect to the nerve. The separate and/or collective closing force of the retainers 14a, 14b is further designed to allow the cuff 10a to be opened with application of a spreading force by a person installing the cuff 10a at the target anatomic feature.

Another distinction between the cuff 10b of FIG. 2 and the cuff 10a of FIG. 1 is that, in an embodiment, the cuff 10b is a tri-polar cuff electrode including three electrodes 16 (shown as electrodes 16a, 16b, 16c) housed within or on an inner circumferential surface of the band 12 at axially spaced-apart locations along the band 12, and the electrical conduits 22 are electrically coupled to the three electrodes 16a, 16b, 16c. by way of example in the embodiment illustrated in FIG. 2, the electrode 16a is shown as having a positive polarity and the electrodes 16b, 16c are each shown as having a negative polarity. Use of three electrodes 16a, 16b, 16c can serve to reduce leakage of electrical energy outside of band 12, and thus also reduce unwanted stimulation outside of the target treatment site.

FIG. 4 is a section view of an embodiment of the cuff 10b of FIG. 2 (e.g., along section line B-B in FIG. 2), similar to the embodiment of the cuff 10a illustrated in FIG. 3, and with the three electrodes 16a, 16b, 16c along the inner circumferential surface of the band 12.

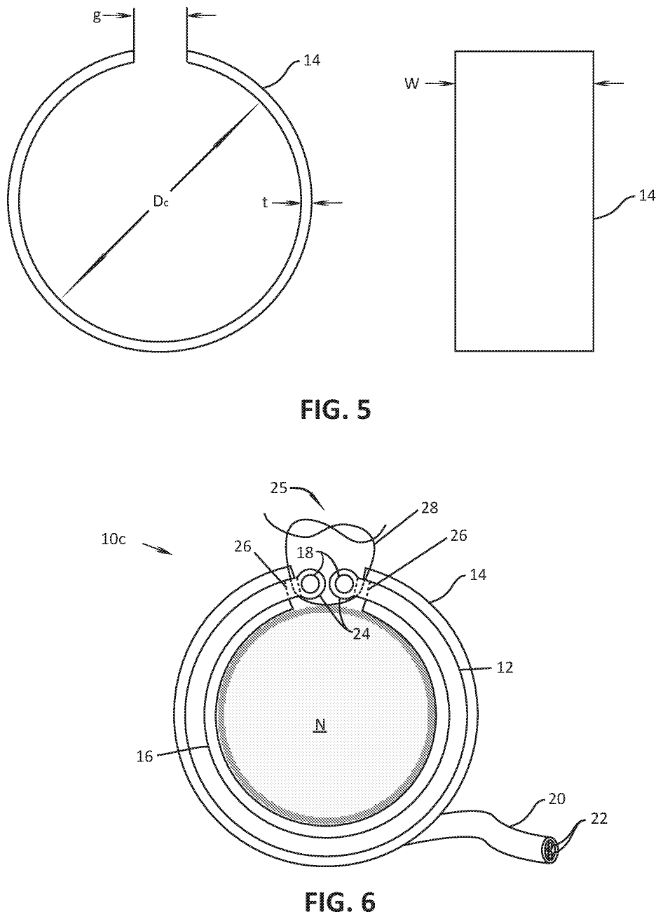

FIG. 5 illustrates an embodiment of the retainer 14 (e.g., the retainer 14 as illustrated in FIG. 1 and FIG. 3, or one or both of the retainers 14a and 14b illustrated in FIG. 2 and FIG. 4). Dimensions of the retainer 14 may be varied to accommodate a size and/or shape of the target anatomic feature as well as a desired closure force of the retainer 14. For example, one or more dimensions of the retainer 14 (e.g., an inner diameter ‘Dc’, a width ‘W’, a circumferential gap width ‘g’, or a thickness ‘t’) may be designed to accommodate the target anatomic feature and to provide a specified closure force of an embodiment of the self-closing cuff 10 incorporating the retainer 14. In an embodiment of the self-closing cuff 10 including one or more electrodes (e.g., one or more of the electrodes 16a, 16b, and/or 16c), the specified closure force of the retainer 14 can provide for sufficient contact between the electrode(s) and an outer surface of the target anatomic feature to maximize a transfer of energy from the electrode(s) into the target anatomic feature.

In an embodiment, one or more retainers 14 (e.g., one or more of the retainers 14, 14a, and/or 14b) are structured to provide a closure force for the self-closing cuff 10 upon release of the spreading force applied to open the circumferential opening 25. Examples of materials that can be used in a retainer 14 to provide the closure force include a metal (e.g., stainless steel), a polymer, a composite, or any other material or combination of materials with sufficient rigidity to retain the retainer 14 (and thus the self-closing cuff 10) in a closed configuration, sufficient flexibility to allow the retainer 14 (and thus the self-closing cuff 10) to be moved into an open configuration, and sufficient resiliency (e.g., memory) to automatically return the retainer 14 (and thus the self-closing cuff 10) to its closed configuration.

In an embodiment, one or more retainers 14 (e.g., one or more of the retainers 14, 14a, and 14b) are structured with multiple components that collectively provide sufficient rigidity to retain the retainer 14 (and thus the self-closing cuff 10) in a closed configuration, sufficient flexibility to allow the retainer 14 (and thus the self-closing cuff 10) to be moved into an open configuration, and sufficient resiliency (e.g., memory) to automatically return the retainer 14 (and thus the self-closing cuff 10) to its closed configuration.

A material of the retainer 14 is generally more rigid than a material of the self-closing cuff 10 to which the retainer 14 is incorporated.

In some situations, a person applying the self-closing cuff 10 may wish to provide a securing mechanism additional to or alternative to a retainer 14 (e.g., additional to or alternative to the retainer 14, 14a, and/or 14b).

FIG. 6 illustrates a self-closing cuff 10c (an embodiment of the self-closing cuff 10), that is similar to the self-closing cuff 10a or the self-closing cuff 10b (e.g., includes a band 12, one or more retainers 14, one or more electrodes 16, longitudinal apertures 18, a sleeve 20, and electrical conduits 22). The cuff 10c is shown installed around a nerve N. The cuff 10c includes a set of suture thru-holes 26 at the opposing ends 24 of the band 12 to allow for a suture 28 to be threaded through the suture thru-holes 26 to secure the circumferential opening 25 in a closed configuration. The suture 28 may be, for example, a thread or a wire or a length of metal, any of which is cut to a desired length before or after use; thus, the suture 28 refers generally to suture material and may refer to a cut length or an uncut length, and multiple sutures may be cut from a single length of suture material. A single set of suture thru-holes 26 may be provided (e.g., at a central location along the longitudinal axis of the band 12), or two or more sets of suture thru-holes 26 may be provided (e.g., at longitudinal ends of the circumferential opening 25). In one example method for fabricating the cuff 10c, the suture thru-holes 26 are generated with a punch to generate approximately 0.035 mm diameter holes through the band 12. Other fabrication means (e.g., injection molding, or extrusion) are also contemplated.

In an embodiment, one or more of the electrodes 16 (e.g., one or more of the electrodes 16a, 16b, and/or 16c) may be attached to the inner circumferential surface of the band 12 via a medical grade adhesive (e.g., MED-2000) or other attachment means. One or more of the electrodes 16 may alternatively be integrated with the band 12. A surface of an electrode 16 exposed within a central channel of the cuff 10 may be co-linear with, protrude from, or be recessed from, the inner circumferential surface of the band 12.

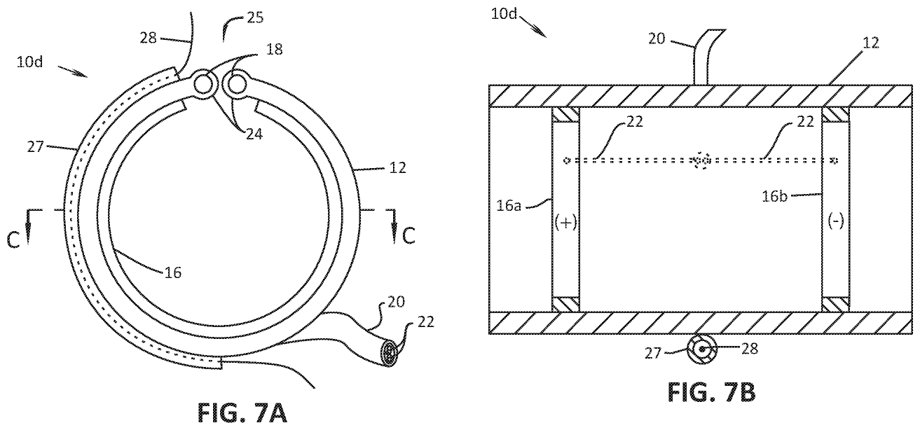

FIG. 7A and FIG. 7B illustrate embodiments of another type of securing mechanism which may be used in addition to, or alternatively to, one or more retainers (e.g., one or more of the retainers 14, 14a, 14b) and/or threading of the suture 28 in the suture thru-holes 26. In the embodiments of FIG. 7A and FIG. 7B, the suture 28 (e.g., similar or different to the suture 28 of FIG. 6) is placed around a self-closing cuff 10d, and ends of the suture 28 are tied together.

FIG. 7A illustrates the self-closing cuff 10d including a suture sleeve or tube 27 in which the suture 28 may be disposed. The tube 27 addresses the situation in which the suture 28 may have a tendency to slip or slide off the cuff, as body fluids often create a lubricous environment. The tube 27 encircles at least a portion of the circumference of the outer surface of the band 12, preferably in a plane orthogonal to the longitudinal axis of the cuff. The tube 27 provides a secured location on the cuff through which the suture 28 can be threaded. When the ends of the suture 28 are tied together (or otherwise coupled), the cuff circumferential opening 25 is secured in a shut configuration (e.g., the opposing ends 24 are restrained from movement away from each other).

In an embodiment, the tube 27 includes a silicone tube (e.g., inner diameter (“ID”)=0.025 inch (or 0.635 mm), outer diameter (“OD”)=0.047 inch (or 1.19 mm)) attached to the band 12, such as via medical grade adhesive (e.g., MED-2000). Alternatively, the tube 27 may be integrated into the cuff 10d wall (e.g., via injection molding or other process), such that a radial channel is created running around a portion of the outer circumference or surface of the band 12.

FIG. 7B is a section view of an embodiment of the cuff 10d of FIG. 7A (e.g., along the section line C-C). The tube 27 may be located to encircle a portion of the cuff outer circumference at a central axial location, as shown in the embodiment of FIG. 7B, or at a different location, or multiple tubes 27 may be located at spaced-apart axial locations.

While the embodiment of the self-closing cuff in FIG. 7B is detailed as a bipolar cuff electrode with two electrodes 16a and 16b on the inner cuff surface, it is appreciated that a tripolar electrode configuration may also be used.

In an embodiment, it is desirable to have an inner diameter of a self-closing cuff (e.g., the cuff 10a, 10b, 10c, or 10d) to be roughly equal to or slightly small than an outer diameter of the target anatomic feature to apply a specified compressive force on the anatomic feature, the specified compressive force being minimized so as not to damage the anatomic feature or alter a nominal function of the target anatomic feature as a result of the specified force. In an embodiment including one more ring electrodes (e.g., one or more of the electrodes 16a, 16b, 16c), an inner diameter of the self-closing cuff may be partially defined by an inner surface of the electrode(s) when the cuff is secured to ensure proper contact between the electrode(s) and the anatomic feature. The term “ring” refers to having a generally arcuate shape (e.g., round) which may or may not be a closed shape as positioned on the anatomic feature (the ends of the ring may or may not touch). Sensitivity of, or tolerance by, an anatomic feature to a compressive force may vary. For example, a nerve, or a lumen susceptible to closure, may tolerate a much smaller force as compared to other anatomic features (e.g., bone, ligaments, or muscles). Further, different persons have differently sized anatomic features. Accordingly, a number of different sized bands (e.g., band 12) and/or a number of different-sized retainers (e.g., retainers 14, 14a, 14b) may be provided in a kit so as to more closely match the anatomic feature for which the cuff is selected. A diagnostic procedure (e.g., imaging procedure such as fluoroscopy, ultrasound, endoscopic image capture, CT, MRI, or other imaging technique) may be performed to estimate a size of the target anatomic feature prior to installation of the cuff so that an appropriate retainer and/or band can be selected.

In an embodiment, the inner diameter of the cuff is somewhat larger than the diameter of the target anatomic feature.

A test was performed to evaluate cuff closure force and equivalent cuff pressure of a cuff designed similarly to cuff 10a of FIGS. 1 and 3 at varying cuff inner diameters and varying cuff circumferential opening 25 distances, with the results of the test shown in Table 1 below. The cuff included a retainer (e.g., an embodiment of the retainer 14 as shown in FIG. 5) formed from a flat ribbon 304V stainless still wire having thickness t=0.002 inch (or 0.051 mm) and width W=0.040 inch (or 1.016 mm).

TABLE 1
Cuff Closure Force and Equivalent Cuff Pressure

| | Edge | | | Equivalent |
|---|---|---|---|---|
| Nominal | Cuff | Expanded | Closure | Cuff |
| Cuff ID | Opening | ID | Force | Pressure |
| (mm) | (mm) | (mm) | (lbf)/(N) | (mmHg) |
| 3 | 0.42 | 3.21 | 0.054/0.240 | 58 |
| | 3.05 | 4.525 | 0.134/0.596 | 144 |
| 4 | 0 | 4 | 0.036/0.160 | 29 |
| | 1.26 | 4.63 | 0.059/0.262 | 48 |
| | 3.15 | 6.75 | 0.09/0.400 | 73 |
| 5 | 0 | 5 | 0.0305/0.136 | 20 |
| | 2 | 6 | 0.051/0.223 | 33 |

In the embodiments detailed in FIG. 1 through FIG. 7B, the electrical conduits 22 are coupled to an internal and/or external controller (not shown) for providing electrical stimulation to a target anatomic feature. Electrodes may be used, by way of example, to provide electrical stimulus to a nerve to stimulate the nerve, form a conduction block within all or a portion of the nerve, denervate the nerve, or other treatment modality. In an embodiment for pudendal nerve stimulation, a self-closing cuff (e.g., the self-closing cuff 10a, 10b, 10c,or 10d) is installed at a location on a pudendal nerve or a branch thereof and coupled to a controller for selectable stimulation and/or conduction blocking of the pudendal nerve. Multiple electrodes on one or more nerves may be associated with such use to control urinary tract function (e.g., micturition or continence control), and one or more of the multiple electrodes may be in a form other than a self-closing cuff.

In addition to urinary tract control, electrical stimulation can be used for a variety of other treatments. Electrical stimulus may be used, for example, for treatment to control bladder, sphincter, or pelvic floor muscles, treatment of pain, numbness, or tingling, and treatment of disease or disorders (e.g., stimulation of vagus nerve for treatment of epilepsy or rheumatoid arthritis, or facial nerve stimulation for treatment of facial palsy). Accordingly, a self-closing cuff can be, for example, positioned around a nerve associated with vertebra (e.g., peripheral nerve, or spinal cord), a sacral nerve, a sciatic nerve, or a cranial nerve (e.g., vagus nerve), to perform one or more treatments with respect to the nerve.

Furthermore, a self-closing cuff may be implemented for treatment of anatomic features outside the nervous system. For example, a self-closing cuff may be installed on vasculature within the body, such as arteries or veins, for providing various cardiovascular treatments or diagnostics. In an embodiment, a self-closing cuff may be sized and shaped to encircle or partially encircle an anatomic feature of, or closely coupled to, the heart (e.g., the pulmonary artery), for providing a circumferential conduction block, stimulation, other treatment, or diagnostic at the installed location.

Although the embodiments detailed in FIG. 1 through FIG. 7B are shown with a circular profile for the self-closing cuff, it is appreciated that a self-closing cuff may have another shape, such as, for example, ellipsoid or other profiles having arcuate and/or planar surfaces, in symmetric or asymmetric configurations that are structured to enclose or otherwise form a coupling contact with a surface of the target anatomic feature.

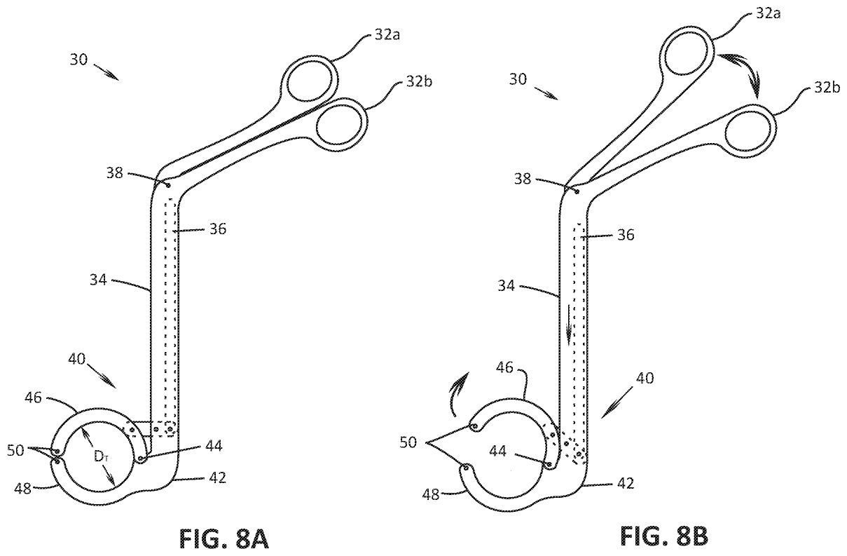

In each of the embodiments in FIG. 1 through FIG. 7B, the band 12 is preferably provided with the pair of longitudinal apertures 18 disposed at ends 24 of circumferential opening 25. The longitudinal apertures 18 preferably extend along a substantial portion of, or the entirety of, the length of the band 12 so as to provide purchase for a placement tool used for providing an expansion force for expanding the circumferential opening 25 during installation and/or removal of the self-closing cuff to/from the target anatomic feature, explained in further detail below with reference to FIG. 8A through FIG. 10. As shown in FIG. 6, FIG. 7A and dashed lines in FIG. 3 and FIG. 4, the ends 24 of the band 12 may also be widened (e.g., via an arcuate projection) to provide a more uniform thickness of material around the longitudinal apertures 18.

In an embodiment, the band 12 includes a compliant but shape-retaining biocompatible material such as urethane, silicone, or the like (e.g., HelixMark silicone medical tubing from Freudenberg Medical Inc.). However, other materials, or material combinations, are contemplated. The band 12 material is preferably sufficiently elastic to be compliant while having sufficient radial spring force to clamp down on and remain on the anatomic feature once installed. Suitable materials include those having a Shore A durometer hardness measurement between 20 A and 60 A, and preferably around 50 A hardness for ease of handling and installation. In an embodiment, band 12 is formed of, or includes, a high consistency silicone elastomer such as MED-4750 available from Avantor. In embodiments provided for nerve stimulation, the dimensions and mechanical and material properties of the assembled cuff (e.g., the cuff 10a, 10b, 10c, or 10d) may be such that the cuff desirably exerts no more than about 150 millimeters (mmHg) of pressure or about 0.5 Newtons of force on an anatomic feature, as forces below this amount may serve to maintain the health of the anatomic feature. The band 12, in particular the inner surface of the band 12, is preferably constructed from atraumatic biomaterials such as one or more of silicone, polyurethane, TEFLON, or the like so as to be atraumatic to the anatomic feature. The inner surface of band 12 may include one more drug eluting coatings to improve the biocompatibility of the self-closing cuff and reduce inflammation or tissue adherence at the interface of the band 12 with the anatomic feature. In a cuff electrode, band 12 is constructed from one or more electrically insulative materials to prevent or minimize possible leaking of electrical current outside of the band 12, and thus prevent or minimize unwanted treatment/stimulation to non-target tissues or anatomic features (e.g., another nerve, a portion or branch of the same nerve, or adjacent tissue).

In one example embodiment of a cuff electrode (e.g., an embodiment of the self-closing cuff 10a, 10b, 10c, or 10d) for providing electrical stimulation to a pudendal nerve, the band 12 includes a silicone tube structure having a Shore A durometer measurement of 50, inner diameter of 2.5 mm, outer diameter of 4 mm and length of 10 mm (e.g., MED-4750).

In an embodiment of a cuff electrode (e.g., an embodiment of the self-closing cuff 10a, 10b, 10c, or 10d), an electrode (e.g., the electrode 16a, 16b or 16c) is about 0.002 inch (or 0.051 mm) thick by about 0.019 inch (or 0.483 mm) wide, in a sheet fabricated from flattened 0.008 inch (or 0.203 mm) diameter platinum wire.

In an embodiment of a self-closing cuff (e.g., an embodiment of the cuff 10a, 10b, or 10c), a retainer (e.g., the retainer 14, 14a, or 14b) is fabricated from 0.002 inch (or 0.051 mm) thick by 0.038 inch (or 0.0.965 mm) wide flat ribbon 304V stainless steel coil with a relaxed or nominal diameter of 2.4 mm-2.6 mm. In another embodiment, a retainer is fabricated from 0.05 mm thick by 1.49 mm wide flat ribbon 316 stainless steel coil with a nominal diameter of 2.66 mm-2.96 mm. In an embodiment, a retainer (e.g., the retainer 14, 14a, or 14b) is cut to have a 3mm gap ‘g’ (see dimension ‘g’ in FIG. 5). A retainer may be attached to a self-closing cuff, such as via a medical grade adhesive (e.g., MED-2000), or may be embedded within the band 12.

While the embodiments disclosed herein are shown with electrodes 16a, 16b and/or 16c that are meant to circumscribe at least a portion of an outer surface of a target anatomic feature, it is appreciated that other electrode configurations may be implemented in combination with or as an alternative to the configurations detailed above and illustrated in FIG. 1 to FIG. 7B. For example, axial electrodes (not shown) may be longitudinally oriented along one or more locations of the inner surface of a band of a self-closing cuff (e.g., the band 12). Helical electrodes (not shown) may also be used when appropriate. The electrodes may also have various shapes and sizes.

Referring now to the illustrations of FIG. 8A to FIG. 10, a method of installation of a self-closing cuff is shown, implementing a placement tool 30 to aid or facilitate installation of a self-closing cuff (e.g., cuff 10a, 10b, 10c, or 10d). For convenience, reference is made to components of the self-closing cuff 10a of FIG. 1; however, the placement tool 30 can also be used with other self-closing cuffs. The placement tool 30 is structured such that the cuff extends perpendicularly from the tool when grasped with the tool.

FIG. 8A shows a side view of the placement tool 30 in a closed configuration, while FIG. 8B shows a side view of the placement tool 30 in an open configuration.

The placement tool 30 includes a pair of crescent (e.g., half-circle) shaped arms 46, 48 joined together at a hinge 44. In the closed configuration (FIG. 8A), the arms 46, 48 come together to form an inner diameter ‘D_{T}’that correlates to (e.g., is equal to or somewhat larger than) a diameter of an outer surface of the target anatomic feature (e.g., an outer diameter of a nerve at the target treatment location), and/or to a diameter of an outer surface of the cuff to be positioned by the placement tool 30. A pin 50 is disposed on an end of each of the arms 46, 48 (e.g., press fit into the arms) and extend orthogonally outward from the arms 46, 48, substantially parallel to an axis of articulation of the arms 46, 48 (e.g., approximately parallel to an axis through hinge 44). Each pin 50 mates to a longitudinal aperture 18 of the cuff 10a, providing an elongated mating surface between the pin 50 and the longitudinal aperture 18. The elongated mating surface distributes a spreading force applied by the placement tool 30 to open the cuff 10a.

The arms 46, 48 are disposed on a distal end 42 of an elongate member 34, which allows the distal end 42 to be inserted into the body to access anatomic features that are difficult to reach from a surface of the body. A pair of handles 32a and 32b are pivotably coupled at a hinge 38 at a proximal end of elongate member 34, and are operatively coupled to a proximal end of a slideable extender 36, the distal end of which is coupled to a linkage 40. Upon extension of the handle 32a with respect to handle 32b, the extender 36 translates with respect to the elongate member 34, causing the linkage 40 to actuate and thus articulate the arm 46 with respect to the arm 48 about the axis of the hinge 44. The articulation of the arm 46 with respect to the arm 48 separates the pins 50. It is appreciated that the linkage 40 may include any mechanism (e.g., gears, cam, or the like mechanism) for converting translation of the extender 36 to a rotation of the arm 46 about the axis of the hinge 44. It is also appreciated that the linkage 40 may convert translation of the extender 36 to rotation of the arm 48 rather than rotation of the arm 46 (with the arm 46 retained in a stationary position by integration with, or fixed attachment to, the elongate member 34), or to rotation of both of the arms 46, 48 (with neither of arms 46, 48 retained in a stationary position).

The placement tool 30 may be biased to the closed orientation shown in FIG. 8A via a spring or multiple springs at each of one or more locations (e.g., at one or both of the hinges 38, 44, or at locations of the linkage 40 or the extender 36). However, a bias of the placement tool 30 may not be needed, particularly where a retainer 14 is used, which serves to naturally bias the cuff 10a in a closed orientation (“self-closing”). In this case, the placement tool 30 may be held closed by the bias of the cuff 10a when the longitudinal apertures 18 of the cuff 10a are engaged with the pins 50 of the placement tool 30.

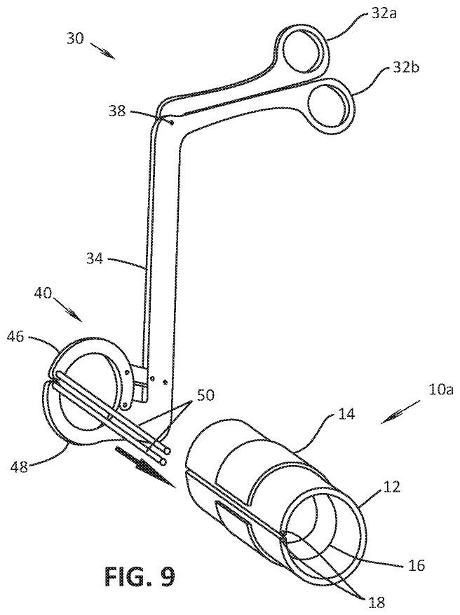

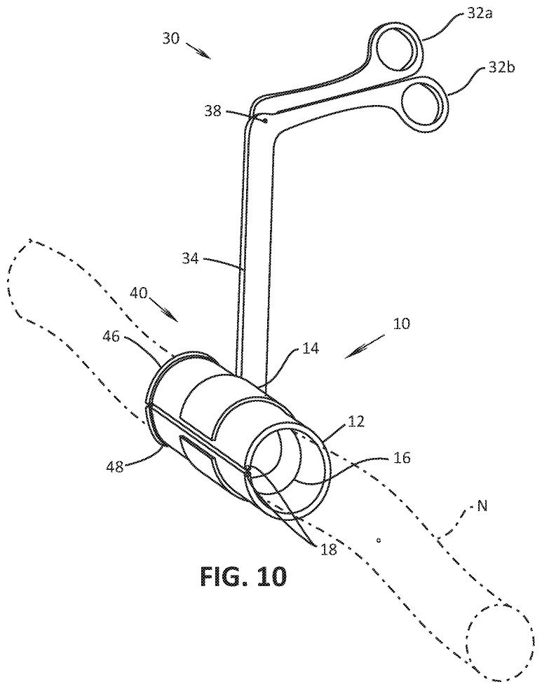

FIG. 9 and FIG. 10 illustrate an embodiment of use of the placement tool 30 of FIGS. 8A, 8B. FIG. 9 illustrates alignment of the placement tool 30 with the cuff 10a prior to engagement, and FIG. 10 illustrates the placement tool 30 and the cuff 10a as engaged and with the cuff 10a positioned over an anatomic feature. Refer to FIG. 1 and FIG. 3 with respect to referenced components of the cuff 10a in the descriptions of FIG. 9 and FIG. 10. Refer to FIGS. 8A, 8B with respect to referenced components of the placement tool 30 in the descriptions of FIG. 9 and FIG. 10.

In FIG. 9, a perspective view is provided of the self-closing cuff 10a prior to being received on the placement tool 30. In an embodiment of a method of installation, the arms 46, 48 of the placement tool 30 are in a closed configuration, and the pins 50 are sized and spaced to match a spacing of the longitudinal apertures 18 of the band 12 when in its natural, resting state. The placement tool 30 is placed such that the pins 50 are in alignment with the longitudinal apertures 18, and the cuff 10a is then translated with respect to the placement tool 30 (as shown by the arrow in FIG. 9) to engage the pins 50 with the longitudinal apertures 18. The pins 50 are thus received in the longitudinal apertures 18 and extend into the cuff 10a at least along a portion of a length of the band 12.

The cuff 10a/placement tool 30 assembly are then advanced within the body such that the distal end 42 of the placement tool 30 is located in proximity to the target treatment location at the target anatomic feature (e.g., a nerve, which may be, for example one of the pudendal nerves or a branch thereof).

The handles 32a and 32b of the placement tool 30 are then spread apart (see FIG. 8B), which causes opening of the arms 46, 48 which in turn causes the pins 50 to apply a spreading force on the longitudinal apertures 18. The applied spreading force causes the circumferential opening 25 to increase. Such increase may be dictated by an available range of articulation of the placement tool 30, or by preference of the user. The placement tool 30 is structured such that the available range of articulation is generally a distance that allows the cuff 10a (or other embodiment) to be advanced toward and over the target anatomic feature. For example, in the instance of installation of the cuff 10a over a nerve, the circumferential opening 25 may be increased to be slightly larger than a diameter of the nerve. In some cases in which an anatomic feature is amenable to a momentary manipulation to squeeze into the confines (e.g., central channel) of the cuff 10a, the circumferential opening 25 may be increased to less than a dimension of the anatomic feature.

As shown in FIG. 10, after positioning the cuff 10a over the anatomic feature (here, the nerve N), the handles 32a, 32b of the placement tool 30 are then brought together, which causes the arms 46, 48 to close, and thus decreases the circumferential opening 25 of the band 12 (e.g., to its natural or unbiased state). If desired, sutures can be applied to further secure the cuff 10a in place. For example, a suture may be wrapped around the cuff 10a, or a suture may be threaded through provided suture thru-holes or tube (e.g., such as in the embodiment shown in FIG. 6 including suture thru-holes 26 in the ends 24 of the band 12, or such as in the embodiment shown in FIG. 7A, 7B including the tube 27 affixed to the band 12).

The placement tool 30 is moved longitudinally to withdraw the pins 50 from the longitudinal apertures 18. To retract the placement tool 30 from the body, the handles 32a, 32b are moved apart and correspondingly the arms 46, 48 are opened, and the placement tool 30 is moved away from the anatomic feature. Optionally, handles 32a, 32b may be moved together such that the placement tool 30 is in a closed configuration before withdrawing the placement tool 30 from the body. The cuff 10a may then remain in place at the location where it was applied for an indefinite period of time.

It is appreciated that while the placement tool 30 is shown in FIG. 8A through 10 to interface with the longitudinal apertures 18 in the band 12, other engagement structures are also within the scope of the present disclosure. For example, an engagement structure can include an engagement surface such as a groove, slot, notch, hook, or other surface structured to provide purchase to the placement tool 30 in addition or alternatively to the longitudinal apertures 18. The engagement surface may be on a band or a retainer of a cuff (e.g., on the band 12 or the retainer 14, 14a, or 14b on the cuff 10a, 10b, 10c, or 10d). For example, the placement tool 30 may be structured to interface with an engagement surface in or on a retainer, such as longitudinal apertures similar to the longitudinal apertures 18 of the cuff 10a, or such as a groove, slot, notch, hook, or other engagement surface, to effect opening of the associated cuff.

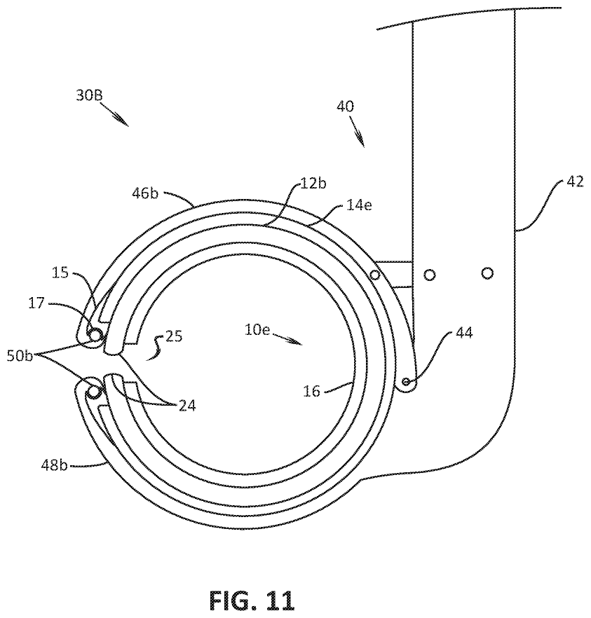

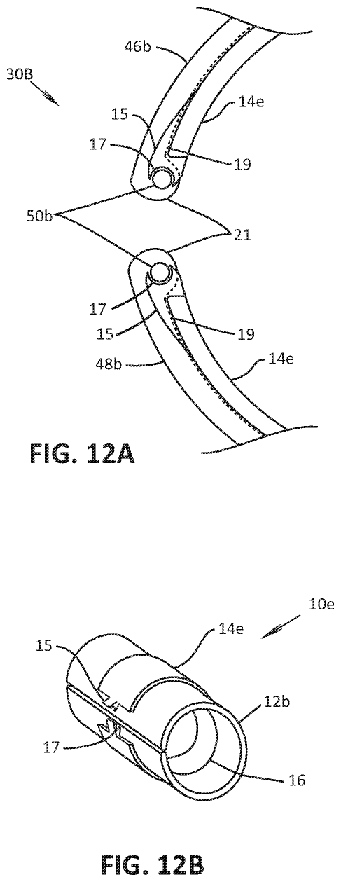

FIG. 11, FIG. 12A, and FIG. 12B illustrate embodiments of a self-closing cuff 10e with alternative engagement surfaces, and a placement tool 30B structured to mate with the cuff 10e. The placement tool 30B is mated to the cuff 10e at a retainer 14e (as shown in an expanded side view of the placement tool 30B coupled to the retainer 14e in FIG. 12A). Arms 46b, 48b of the placement tool 30B have a collective inner surface 19 (FIG. 12A) structured such that dimensions of the inner surface 19 (e.g., a diameter of the inner surface 19) are equal to or slightly greater than dimensions of the retainer 14e (e.g., a diameter of an outer surface of the retainer 14e). The placement tool 30B is structured with engagement surfaces including a pair of pins 50b disposed at ends 21 of arms 46b, 48b of the placement tool 30B. The retainer 14e is structured with engagement surfaces including a pair of channels 17 disposed at thickened central tabs 15 of the retainer 14e (FIG. 12B). The pins 50b of the placement tool 30B can slide longitudinally along the channels 17 of the retainer 14e. The retainer 14e is adhered to an outer surface of a band 12b, or is integrated with or into the band 12b (e.g., via adhered layers, injection molding, or other process) so that the band 12b and the retainer 14e move in concert when a spreading force is applied to the retainer 14e as a result of engagement with the placement tool 30B.

In an embodiment, the self-closing cuff 10e is a cuff electrode including one or more electrodes (e.g., electrode 16a, 16b, and/or 16c). In a method of treatment, a controller (not shown) may be coupled to the cuff 10e via electrical conduits, either prior to or after placement, and then operated to apply a signal to the one or more electrodes for treatment or therapy to the target anatomic feature (e.g., nerve N). In the case of pudendal nerve stimulation, the applied signal includes an electrical signal that either stimulates or forms a conduction block of the pudendal nerve or a branch thereof to cause an opening/closing of the urethral sphincter or contraction/relaxation of a detrusor muscle.

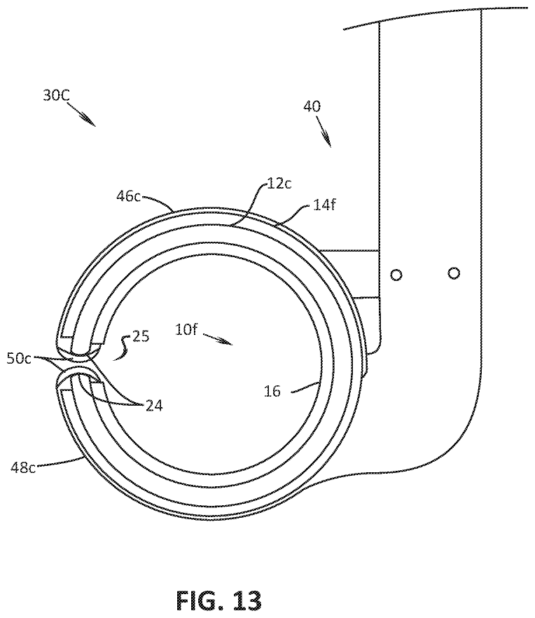

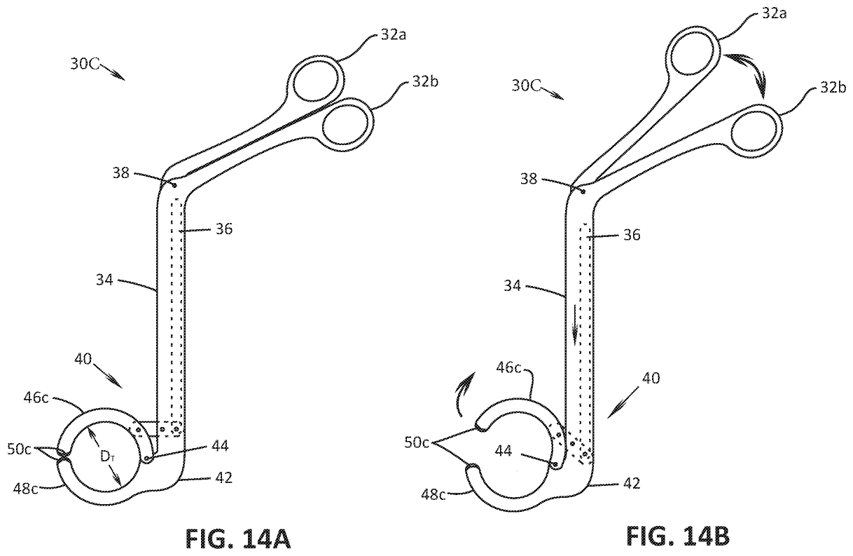

FIG. 13, FIG. 14A, and FIG. 14B illustrate embodiments of a placement tool 30C structured to mate with a self-closing cuff 10f with alternative engagement surfaces. Instead of the pins 50 detailed in the placement tool 30 of FIG. 8A and 8B, the placement tool 30C includes a pair of spreading surfaces in the form of cups 50c disposed at ends of arms 46c, 48c. In an embodiment, the spreading surfaces 50c include opposing concave curved surfaces spaced and oriented to engage ends 24 of a band 12c. The cupped surfaces 50c extend perpendicularly from the arms 46c, 48c along a rotation axis (e.g., an axis through a hinge similar to the hinge 44 in FIGS. 8A, 8B), and have a length corresponding to a length of the band 12c or corresponding to less than the length of the band 12c (e.g., less than 70% of the length of the band 12c, or less than 50% of the band 12c). A spreading force can be applied by the cupped surfaces 50c against the ends 24 of the band 12c with the force distributed along the length of the cupped surfaces 50c. FIG. 14A illustrates the placement tool 30C in a closed configuration, and FIG. 14B illustrates the placement tool 30C in an open configuration.

It is appreciated that mating surfaces other than described with respect to FIG. 1-FIG. 14B can provide engagement between a self-closing cuff and a placement tool. Additionally, components of the placement tool having the mating surfaces of the placement tool may be removably attached to the placement tool such that different components with different mating surfaces may be attached. Examples of mating surfaces generally, whether or not removable from the placement tool, include a component with a convex surface (and the corresponding surface of the cuff being concave), or a component having a rectangular, V, T, or other shape channel (with a corresponding mating shape on the cuff).

In an embodiment, any one or more of the electrodes 16 (e.g., the electrodes 16a, 16b, and/or 16c) are ring electrodes (e.g., have an arcuate profile that follows a profile of an inner circumferential surface of the band 12).

The self-closing cuffs of the present disclosure (e.g., cuffs 10a, 10b, 10c, 10d, 10e, 10f) are biocompatible, using biocompatible materials and/or shielding one or more components with a shield formed of biocompatible materials.

In one or more applications, a self-closing cuff (e.g., the cuff 10a, 10b, 10c, 10d, 10e, or 10f), a placement tool (e.g., the placement tool 30, 30B, or 30C), and corresponding methods of installing the cuff via the placement tool and methods of treatment, are particularly beneficial for treatment/therapy of a target treatment location or region that may be adjacent to or at a curved anatomic feature, which may be in a thin, elongate or otherwise tubular or arcuate form. Such target anatomic features may include nerves, arteries or other vasculature, luminal organs, bones, other tissues such as tendons, ligaments, musculature, or fascia, or other features.

It is appreciated that the configurations provided in this disclosure are provided for example purposes only, and dimensions and material selection may vary according to application and preference.

Embodiments of the present disclosure include improved systems and methods for providing electrical stimulation to a location in or on the body. In particular, the systems and method disclosed herein are directed to a self-closing cuff, along with methods and devices for installation at a target treatment or diagnostic location of the body. Embodiments of the present disclosure include improved systems and methods for retaining a device at a location in or on the body. In particular, the systems and method disclosed herein are directed to a self-closing cuff, along with methods and devices for installation at a target location. From the description herein, it will be appreciated that the present disclosure encompasses multiple embodiments which include, but are not limited to, the following:

1. An implant for providing medical treatment to a treatment location of a target anatomic feature within the body, the target anatomic feature including a curved outer surface, the implant including: a tubular cuff having a central channel defined by an inner surface of the cuff, the cuff including a circumferential opening spanning longitudinally along a length of the cuff to form a c-shaped profile at least partially circumscribing the central channel; one or more electrodes disposed on the inner surface of the cuff, the one or more electrodes structured to deliver an electrical signal to provide treatment at the treatment location of the target anatomic feature; and a retainer structured to secure around at least a portion of an outer surface of the cuff; wherein the circumferential opening is coupled to an engagement surface for application of a spreading force to be applied to the cuff to expand to circumferential opening from a closed configuration to an open configuration sized for placement of the cuff at the target anatomic feature; and wherein the cuff is self-closing such that upon release of the spreading force, the circumferential opening automatically closes toward the closed configuration, the closed configuration ensuring contact between the one or more electrodes and curved surface of the target anatomic feature.

2. The implant, system or method of any of the preceding or subsequent embodiments: wherein the retainer includes a retainer disposed around the cuff; and wherein the retainer is sized, shaped and composed of a material structured to enhance self-closing of the cuff.

3. The implant, system or method of any of the preceding or subsequent embodiments: wherein the retainer includes a suture channel disposed on an outer surface of the cuff; the channel running at least partially along the outer surface in a direction substantially normal to circumferential opening; the suture channel sized to retain a suture for securing closure of the circumferential opening upon installation of the cuff to the target anatomic feature at the treatment location.

4. The implant, system or method of any of the preceding or subsequent embodiments: wherein the cuff includes a pair of apertures running through the cuff at locations proximal to the circumferential opening of the cuff, the pair of apertures providing clearance for a suture.

5. The implant, system or method of any of the preceding or subsequent embodiments: wherein the engagement surface includes a pair of longitudinal apertures running along at least a portion of a length of the cuff, the pair of longitudinal apertures being disposed at opposite sides of the circumferential opening.

6. The implant, system or method of any of the preceding or subsequent embodiments: the one or more electrodes include a pair of axially spaced-apart electrodes housed within or on an inner circumferential surface of the cuff at axially spaced-apart locations the cuff to form a bi-polar electrode configuration.

7. The implant, system or method of any of the preceding or subsequent embodiments: wherein the one or more electrodes include a first electrode having a first pole and second and third electrodes having a second pole, the first, second and third electrodes being housed at axially spaced-apart locations within or on the inner circumferential surface to form a tri-polar electrode configuration.

8. The implant, system or method of any of the preceding or subsequent embodiments: wherein the central channel includes an inner circumferential surface sized and shaped to couple to a target anatomic feature including a nerve, wherein the one or more electrodes contact an outer surface of the nerve at spaced apart locations to provide a stimulation or conduction-block electrical signal to the nerve.

9. A system for installing a cuff to a treatment location of a target anatomic feature within the body, the target anatomic feature including a curved outer surface, the system including: a cuff including a tubular electrode cuff having a central channel defined by an inner surface of the cuff, the cuff including a circumferential opening spanning longitudinally along a length of the cuff to form a c-shaped profile at least partially circumscribing the central channel, and one or more electrodes disposed on the inner surface of the cuff, the one or more electrodes structured to deliver an electrical signal to provide treatment at the treatment location of the target anatomic feature, the cuff being biased in a closed configuration, the cuff further including an engagement surface; and a placement tool structured to interface with the engagement surface of the cuff; the placement tool structured to apply a spreading force to the engagement surface while the circumferential opening is in the closed configuration, and expand the circumferential opening, by way of the spreading force, from the closed configuration to an open configuration sized for placement of the cuff on to the target anatomic feature, position the cuff such that the central channel at least partially encircles the target anatomic feature, and articulate the placement tool to release the spreading force on the engagement surface; wherein the cuff is self-closing such that upon release of the spreading force, the circumferential opening automatically closes toward the closed configuration to affect contact between the one or more electrodes and curved surface of the target anatomic feature.

10. The implant, system or method of any of the preceding or subsequent embodiments: the cuff further including a retainer: wherein the retainer includes a retainer disposed around the cuff; and wherein the retainer is sized, shaped and composed of a material structured to enhance self-closing of the cuff.

11. The implant, system or method of any of the preceding or subsequent embodiments: the cuff further including a retainer: wherein the retainer includes a suture channel disposed on an outer surface of the cuff; the channel running at least partially along the outer surface in a direction substantially normal to circumferential opening; the suture channel sized to retain a suture for securing closure of the circumferential opening upon installation of the cuff to the target anatomic feature at the treatment location.

12. The implant, system or method of any of the preceding or subsequent embodiments: wherein the cuff includes a pair of apertures running through the cuff at locations proximal to the circumferential opening of the cuff, the pair of apertures providing clearance for a suture.

13. The implant, system or method of any of the preceding or subsequent embodiments: wherein the engagement surface includes a pair of longitudinal apertures running along at least a portion of a length of the cuff, the pair of longitudinal apertures being disposed at opposite sides of the circumferential opening; and wherein the placement tool includes a pair of articulating pins structured to be slideably received in the pair of longitudinal apertures.

14. The implant, system or method of any of the preceding or subsequent embodiments: wherein the pair of pins are disposed on an articulating pair of arms, the pair of arms when in a closed configuration forming a profile substantially matching the c-shaped profile of the cuff.

15. The implant, system or method of any of the preceding or subsequent embodiments: wherein the one or more electrodes include a pair of axially spaced-apart electrodes housed within or on the inner surface of the cuff at axially spaced-apart locations of the cuff to form a bi-polar electrode configuration.

16. The implant, system or method of any of the preceding or subsequent embodiments: wherein the one or more electrodes include a first electrode having a first pole and second and third electrodes having a second pole, the first, second and third electrodes being housed at axially spaced-apart locations within or on the inner surface of the cuff to form a tri-polar electrode configuration.

17. The implant, system or method of any of the preceding or subsequent embodiments: wherein the central channel includes an inner circumferential surface sized and shaped to couple to a target anatomic feature including a nerve, wherein the one or more electrodes contact an outer surface of the nerve at spaced apart locations to provide a stimulation or conduction-block electrical signal to the nerve.

18. A method for installing a cuff to a treatment location of a target anatomic feature within the body, the target anatomic feature including a curved outer surface, the method including: providing a cuff including a tubular electrode cuff having a central channel defined by an inner surface of the cuff, the cuff including a circumferential opening spanning longitudinally along a length of the cuff to form a c-shaped profile at least partially circumscribing the central channel, and one or more electrodes disposed on the inner surface of the cuff, the one or more electrodes structured to deliver an electrical signal to provide treatment at the treatment location of the target anatomic feature, the cuff being biased in a closed configuration, the cuff further including an engagement surface; coupling a placement tool to the engagement surface of the cuff; articulating the placement tool to apply a spreading force to the engagement surface starting while the circumferential opening is in the closed configuration; expanding the circumferential opening, by way of the spreading force; from the closed configuration to an open configuration sized for placement of the cuff on to the target anatomic feature; positioning the cuff such that the central channel at least partially encircles the target anatomic feature; and articulating the placement tool to release the spreading force on the engagement surface; wherein the cuff is self-closing such that upon release of the spreading force, the circumferential opening automatically closes toward the closed configuration to affect contact between the one or more electrodes and curved surface of the target anatomic feature.

19. The implant, system or method of any of the preceding or subsequent embodiments: the cuff further including a retainer.

20. The implant, system or method of any of the preceding or subsequent embodiments: wherein the retainer includes a retainer disposed around the cuff; and wherein the retainer is sized, shaped and composed of a material structured to enhance self-closing of the cuff.

21. The implant, system or method of any of the preceding or subsequent embodiments: wherein the retainer includes a suture channel disposed on an outer surface of the cuff; the channel running at least partially along the outer surface in a direction substantially normal to circumferential opening, the method further including: inserting a suture into the suture channel; and tying the suture to secure closure of the circumferential opening after installation of the cuff to the target anatomic feature at the treatment location.

22. The implant, system or method of any of the preceding or subsequent embodiments: wherein the cuff includes a pair of apertures running through the cuff at locations proximal to the circumferential opening of the cuff, the method further including: inserting a suture through the pair of apertures; and tying the suture to secure closure of the circumferential opening after installation of the cuff to the target anatomic feature at the treatment location

23. The implant, system or method of any of the preceding or subsequent embodiments: wherein the engagement surface includes a pair of longitudinal apertures running along at least a portion of a length of the cuff, the pair of longitudinal apertures being disposed at opposite sides of the circumferential opening, wherein coupling a placement tool to an engagement surface of the cuff includes: slideably receiving a pair of articulating pins into the pair of longitudinal apertures.

24. The implant, system or method of any of the preceding or subsequent embodiments: wherein the pair of pins are disposed on an articulating pair of arms, the pair of arms when in a closed configuration forming a profile substantially matching the c-shaped profile of the cuff.

25. The implant, system or method of any of the preceding or subsequent embodiments: wherein the one or more electrodes include a pair of axially spaced-apart electrodes housed within or on the inner surface of the cuff at axially spaced-apart locations of the cuff to form a bi-polar electrode configuration.

26. The implant, system or method of any of the preceding or subsequent embodiments: wherein the one or more electrodes include a first electrode having a first pole and second and third electrodes having a second pole, the first, second and third electrodes being housed at axially spaced-apart locations within or on the inner surface of the cuff to form a tri-polar electrode configuration.

27. The implant, system or method of any of the preceding or subsequent embodiments: wherein the central channel includes an inner circumferential surface sized and shaped to couple to a target anatomic feature including a nerve, the method further including: contacting the one or more electrodes to an outer surface of the nerve at spaced apart locations; sending at least one electrical signal through the one or more electrodes for stimulation or conduction-block of the nerve.

28. The implant, system or method of any of the preceding or subsequent embodiments: wherein the nerve includes a pudendal nerve or a branch thereof, and wherein the stimulation or conduction-block of the pudendal nerve is to cause at least a first and second physiological action involved in control of the bladder.

29. A cuff provides for medical treatment to a treatment location of a target anatomic feature within the body, where the target anatomic feature includes a curved outer surface. The cuff includes: a band defining a circumferential opening extending along a length of the band; a pair of engagement surfaces defined by or affixed to the cuff, the engagement surfaces structured for application of a spreading force to be distributed continuously along a portion of the circumferential opening, thereby increasing the circumferential opening and accordingly expanding the cuff from a closed configuration to an open configuration sized for placement of the cuff at the target anatomic feature. The cuff is structured to remain in the closed configuration in the absence of the spreading force, and structured to automatically return to the closed configuration after removal of the spreading force.

30. The cuff may include a retainer disposed along at least a portion of the circumference of the cuff, the retainer and the cuff being structured to move in concert with respect to each other during expansion of the circumferential opening upon application of the spreading force. The engagement surfaces may be defined by or affixed to the retainer, or the engagement surfaces may be defined by or affixed to the band. The cuff may be structured to apply a compressive force of less than 150 mmHg of pressure to the target anatomic feature in the closed configuration of the cuff. The cuff may have one or more electrodes disposed on an inner surface of the cuff. The engagement surfaces may be a pair of longitudinal apertures extending along a length of the cuff, or opposing protrusions or channels extending along a length of the cuff. The cuff may be structured in the closed configuration to ensure contact between the one or more electrodes and the curved surface of the target anatomic feature. The target anatomic feature may be a nerve, an inner surface of the cuff defines a central channel sized and shaped to couple to the nerve, and the cuff further includes one or more electrodes structured to contact an outer surface of the nerve to deliver an electrical signal to the nerve.

31. A system is provided for installing a cuff to a treatment location of a target anatomic feature within the body, where the target anatomic feature includes a curved outer surface. The system includes: a cuff defining a circumferential opening spanning longitudinally along a length of the cuff, the cuff being in a closed configuration in a relaxed state, the cuff including a pair of opposing engagement surfaces; and a placement tool structured to interface with the engagement surfaces of the cuff. The placement tool is structured to: be articulated to apply a spreading force to the engagement surfaces while the cuff is in the closed configuration to increase the circumferential opening such that the cuff expands from the closed configuration to an open configuration sized for placement of the cuff on the target anatomic feature; be positioned while the cuff is in the open configuration such that the cuff at least partially encircles the target anatomic feature; and be articulated to release the spreading force on the engagement surfaces while the cuff at least partially encircles the target anatomic feature. The cuff is self-closing such that upon release of the spreading force, the cuff automatically returns to the closed configuration.

32. The system may include one or more electrodes disposed on an inner surface of the cuff, the one or more electrodes structured to deliver an electrical signal to the target anatomic feature. The system may include a band and a retainer secured along at least a portion of the band, wherein the retainer and the band are sized, shaped and composed of a material structured to allow for an increase of the circumferential opening upon application of the spreading force. The placement tool may include a pair of opposing spreading surfaces structured to couple to the engagement surfaces of the cuff for distribution of the spreading force along the engagement surfaces; and the spreading surfaces are disposed on respective arms of an articulating pair of arms, the pair of arms when in a closed configuration forming an inner profile substantially matching an outer profile of the cuff. The engagement surfaces may include a pair of longitudinal apertures running along a length of the cuff, the pair of longitudinal apertures being disposed at opposite sides of the circumferential opening; and the spreading surfaces may include a pair of articulating pins structured to be slideably received in the pair of longitudinal apertures and contact the engagement surfaces for application of the spreading force. The engagement surfaces may include a pair of channels or protrusions disposed at opposite sides of the circumferential opening of the cuff; and the spreading surfaces may include a pair of mating surfaces structured to contact the engagement surfaces for application of the spreading force. The spreading surfaces may be structured to be slideably received into the circumferential opening to then be mated with the engagement surfaces. The system may further include one or more electrodes within or on an inner surface of the cuff to form a bi-polar or tri-polar electrode configuration.

33. A method is provided for installing a cuff to a treatment location of a target anatomic feature within a body. The method includes: providing a cuff defining a circumferential opening extending longitudinally along a length of the cuff, the cuff in its natural relaxed state being in a closed configuration, the cuff including a pair of engagement surfaces; coupling a placement tool to the engagement surfaces of the cuff; articulating the placement tool while the cuff is in the closed configuration to increase the circumferential opening by way of a spreading force such that the cuff expands from the closed configuration to an open configuration sized for placement of the cuff on the target anatomic feature; positioning the cuff such that the cuff at least partially encircles the target anatomic feature; articulating the placement tool to release the spreading force on the engagement surfaces. The cuff is self-closing such that upon release of the spreading force, the cuff automatically returns to the closed configuration; and disengaging the placement tool from the cuff.

34. The disengaging of the placement tool from the cuff may include decoupling the placement tool from the engagement surfaces of the cuff, articulating the placement tool to increase a dimension of an inner surface defined by a pair of arms of the placement tool, and extricating the placement tool from a position at least partially encircling the target anatomic feature to a position external to the body. The cuff may further include one or more electrodes disposed on an inner surface of the cuff, the one or more electrodes structured to deliver an electrical signal to the target anatomic feature. The cuff may further include a band and a retainer secured along at least a portion of a circumference of the band; wherein the retainer and the band are sized, shaped and composed of materials structured to allow for the increase in the circumferential opening upon application of the spreading force, and further structured to apply a specified force to the target anatomic feature in the closed configuration. The placement tool may include a pair of opposing spreading surfaces structured to couple to the engagement surfaces of the cuff for contiguous distribution of the spreading force along the engagement surfaces. The spreading surfaces may be disposed on respective arms of an articulating pair of arms, the pair of arms when in a closed configuration having an inner surface profile substantially matching an outer surface profile of the cuff. The engagement surfaces may include a pair of longitudinal apertures running along at least a portion of a length of the cuff and the spreading surfaces may include a pair of pins. Coupling a placement tool to the engagement surfaces of the cuff may include: slideably receiving the pair of pins in respective longitudinal apertures; and applying the spreading force contiguously along a longitudinal length of the engagement surfaces. The engagement surfaces may include a pair of channels or protrusions disposed at opposite sides of the circumferential opening and the spreading surfaces may include a pair of mating surfaces. The method may include slideably receiving the spreading surfaces into the circumferential opening; contacting the engagement surfaces with the spreading surfaces; and applying the spreading force contiguously along a longitudinal length of the engagement surfaces.

In any of the foregoing embodiments, the self-closing cuff may be attached to a housing of an implantable medical treatment device, or may be attached to a catheter or cannula.

The foregoing description of various embodiments of the technology of the present disclosure has been presented for purposes of illustration and description. It is not intended to limit the technology of the present disclosure to the precise forms disclosed. Many modifications, variations and refinements will be apparent to practitioners skilled in the art. For example, embodiments of the device can be sized and otherwise adapted for various pediatric and neonatal applications as well as various veterinary applications. They may also be adapted for the urinary tracts of both males and females. Further, those skilled in the art will recognize, or be able to ascertain using no more than routine experimentation, numerous equivalents to the specific devices and methods described herein. Such equivalents are considered to be within the scope of the technology of the present disclosure and are covered by the appended claims below.

Elements, characteristics, or acts from one embodiment can be readily recombined or substituted with one or more elements, characteristics or acts from other embodiments to form numerous additional embodiments within the scope of the technology of the present disclosure. Moreover, elements that are shown or described as being combined with other elements, can, in an embodiment, exist as standalone elements. Hence, the scope of the technology of the present disclosure is not limited to the specifics of the described embodiments, but is instead limited solely by the appended claims.

What is claimed is:

1. A cuff for providing medical treatment to a treatment location of a target anatomic feature within the body, the target anatomic feature comprising a curved outer surface, the cuff comprising:
a band defining a circumferential opening extending along a length of the band; and
a pair of engagement surfaces defined by or affixed to the cuff, the engagement surfaces structured for application of a spreading force to be distributed continuously along a portion of the circumferential opening, thereby increasing the circumferential opening and accordingly expanding the cuff from a closed configuration to an open configuration sized for placement of the cuff at the target anatomic feature;
wherein the cuff is structured to remain in the closed configuration in the absence of the spreading force, and structured to automatically return to the closed configuration after removal of the spreading force.

2. The cuff of claim 1, further comprising a retainer disposed along at least a portion of the circumference of the cuff, the retainer and the cuff being structured to move in concert with respect to each other during expansion of the circumferential opening upon application of the spreading force.

3. The cuff of claim 2, wherein the engagement surfaces are defined by or affixed to the retainer.

4. The cuff of claim 1, wherein the engagement surfaces are defined by or affixed to the band.

5. The cuff of claim 1, wherein the cuff is structured to apply a compressive force of less than 150 mmHg of pressure to the target anatomic feature in the closed configuration of the cuff.

6. The cuff of claim 1, further comprising one or more electrodes disposed on an inner surface of the cuff.

7. The cuff of claim 1, wherein the engagement surfaces comprise a pair of longitudinal apertures extending along a length of the cuff.

8. The cuff of claim 1, wherein the engagement surfaces comprise opposing protrusions or channels extending along a length of the cuff.

9. The cuff of claim 1, wherein the target anatomic feature is a nerve, an inner surface of the cuff defines a central channel sized and shaped to couple to the nerve, and the cuff further comprises one or more electrodes structured to contact an outer surface of the nerve to deliver an electrical signal to the nerve.

10. The cuff of claim 9, wherein the cuff is structured in the closed configuration to ensure contact between the one or more electrodes and the curved surface of the target anatomic feature.

11. A system for installing a cuff to a treatment location of a target anatomic feature within the body, the target anatomic feature comprising a curved outer surface, the system comprising:
a cuff defining a circumferential opening spanning longitudinally along a length of the cuff, the cuff being in a closed configuration in a relaxed state, the cuff comprising a pair of opposing engagement surfaces; and
a placement tool structured to interface with the engagement surfaces of the cuff, the placement tool structured to: be articulated to apply a spreading force to the engagement surfaces while the cuff is in the closed configuration to increase the circumferential opening such that the cuff expands from the closed configuration to an open configuration sized for placement of the cuff on the target anatomic feature; be positioned while the cuff is in the open configuration such that the cuff at least partially encircles the target anatomic feature; and be articulated to release the spreading force on the engagement surfaces while the cuff at least partially encircles the target anatomic feature;
wherein the cuff is self-closing such that upon release of the spreading force, the cuff automatically returns to the closed configuration.

12. The system of claim 11, further comprising one or more electrodes disposed on an inner surface of the cuff, the one or more electrodes structured to deliver an electrical signal to the target anatomic feature.

13. The system of claim 11, the cuff further comprising a band and a retainer secured along at least a portion of the band, wherein the retainer and the band are sized, shaped and composed of a material structured to allow for an increase of the circumferential opening upon application of the spreading force.

14. The system of claim 11, wherein the placement tool comprises a pair of opposing spreading surfaces structured to couple to the engagement surfaces of the cuff for distribution of the spreading force along the engagement surfaces; and wherein the spreading surfaces are disposed on respective arms of an articulating pair of arms, the pair of arms when in a closed configuration forming an inner profile substantially matching an outer profile of the cuff.

15. The system of claim 14:
wherein the engagement surfaces comprise a pair of longitudinal apertures running along a length of the cuff, the pair of longitudinal apertures being disposed at opposite sides of the circumferential opening; and
wherein the spreading surfaces comprise a pair of articulating pins structured to be slideably received in the pair of longitudinal apertures and contact the engagement surfaces for application of the spreading force.

16. The system of claim 14:
wherein the engagement surfaces comprise a pair of channels or protrusions disposed at opposite sides of the circumferential opening of the cuff; and
wherein the spreading surfaces comprise a pair of mating surfaces structured to contact the engagement surfaces for application of the spreading force.

17. The system of claim 16, wherein the spreading surfaces are structured to be slideably received into the circumferential opening to then be mated with the engagement surfaces.

18. The system of claim 11, further comprising one or more electrodes within or on an inner surface of the cuff to form a bi-polar or tri-polar electrode configuration.

19. A method for installing a cuff to a treatment location of a target anatomic feature within a body, the method comprising:
providing a cuff defining a circumferential opening extending longitudinally along a length of the cuff, the cuff in its natural relaxed state being in a closed configuration, the cuff comprising a pair of engagement surfaces;
coupling a placement tool to the engagement surfaces of the cuff;
articulating the placement tool while the cuff is in the closed configuration to increase the circumferential opening by way of a spreading force such that the cuff expands from the closed configuration to an open configuration sized for placement of the cuff on the target anatomic feature;
positioning the cuff such that the cuff at least partially encircles the target anatomic feature;
articulating the placement tool to release the spreading force on the engagement surfaces, wherein the cuff is self-closing such that upon release of the spreading force, the cuff automatically returns to the closed configuration; and
disengaging the placement tool from the cuff.

20. The method of claim 16, wherein disengaging the placement tool from the cuff comprises decoupling the placement tool from the engagement surfaces of the cuff, articulating the placement tool to increase a dimension of an inner surface defined by a pair of arms of the placement tool, and extricating the placement tool from a position at least partially encircling the target anatomic feature to a position external to the body.

21. The method of claim 19, the cuff further comprising one or more electrodes disposed on an inner surface of the cuff, the one or more electrodes structured to deliver an electrical signal to the target anatomic feature.

22. The method of claim 19, the cuff further comprising a band and a retainer secured along at least a portion of a circumference of the band; wherein the retainer and the band are sized, shaped and composed of materials structured to allow for the increase in the circumferential opening upon application of the spreading force, and further structured to apply a specified force to the target anatomic feature in the closed configuration.

23. The method of claim 19, wherein the placement tool comprises a pair of opposing spreading surfaces structured to couple to the engagement surfaces of the cuff for contiguous distribution of the spreading force along the engagement surfaces; and wherein the spreading surfaces are disposed on respective arms of an articulating pair of arms, the pair of arms when in a closed configuration having an inner surface profile substantially matching an outer surface profile of the cuff.

24. The method of claim 23, wherein the engagement surfaces comprise a pair of longitudinal apertures running along at least a portion of a length of the cuff and the spreading surfaces comprise a pair of pins, wherein coupling a placement tool to the engagement surfaces of the cuff comprises:
slideably receiving the pair of pins in respective longitudinal apertures; and
applying the spreading force contiguously along a longitudinal length of the engagement surfaces.

25. The method of claim 23, wherein the engagement surfaces comprise a pair of channels or protrusions disposed at opposite sides of the circumferential opening and the spreading surfaces comprise a pair of mating surfaces; and
slideably receiving the spreading surfaces into the circumferential opening;
contacting the engagement surfaces with the spreading surfaces; and
applying the spreading force contiguously along a longitudinal length of the engagement surfaces.
